(19) United States
(12) Patent Application Publication
(10) Pub. No.: US 2023/0173182 A1
(43) Pub. Date: Jun. 8, 2023

(54) AN AUTO-INJECTOR DEVICE FOR DELIVERING MEDICAMENTS

(71) Applicant: BIOCON BIOLOGICS LIMITED, Karnataka (IN)

(72) Inventors: Jeevan Maddur SHANKARSETTY, Karnataka (IN); Shanthan JANGILI, Telangana (IN); Ayanur Nadig Chetan RAO, Karnataka (IN); Shreehas Pradeep TAMBE, Karnataka (IN); Anu Kumar Haranahalli SADASHIVA, Mysuru (IN)

(73) Assignee: BIOCON BIOLOGICS LIMITED, Karnataka (IN)

(21) Appl. No.: 17/922,691

(22) PCT Filed: May 14, 2021
(86) PCT No.: PCT/IN2021/050463

§ 371 Date: Nov. 1, 2022

(30) Foreign Application Priority Data
May 15, 2020 (IN) 202041020640

(51) Int. Cl. A61M 5/20; A61M 5/32; A61M 5/315

(52) U.S. Cl. CPC A61M 5/2033; A61M 5/321; A61M 5/31501

(57) ABSTRACT

An auto-injector device for delivering medicaments is disclosed. The auto-injector device includes a syringe assembly having a needle adapted to expel medicaments from the syringe assembly and a barrel adapted to hold the medicaments. The syringe assembly includes a plunger positioned coaxially with respect to the barrel and adapted to reciprocate within the barrel to expel the medicaments from the barrel. Further, the syringe assembly includes a locking member adapted to be engaged with the plunger to hold he plunger in a position with respect to the barrel. The position of the plunger with respect to the barrel is adapted to be adjusted to vary a volume of medicaments to be expelled from the barrel. The position of the plunger is adjusted by engaging the locking member with the plunger at one of a plurality of positions along a length of the plunger.

### FIELD OF THE INVENTION

The present disclosure relates to medication delivery devices and more particularly, to an auto-injector device for delivering medicaments.

### BACKGROUND

With the advancement in technology, various injection devices, such as manual injection devices and auto-injector devices, are employed for administering medicaments for applications such as medical, therapeutic, diagnostic, pharmaceutical and cosmetic. Nowadays, users and healthcare professionals are more inclined towards using the auto-injector devices for administering medicaments. One of the prominent reasons for such inclination is a substantial reduction in the amount of training required for using the auto-injector devices.

Generally, an auto-injector device includes a prefilled syringe or cartridge filled with a specific volume of a medicament to be delivered to the user. A volume of the medicament filled in the auto-injector device depends on various parameters, such as a type of medicament and a medical condition of the user. Currently, the auto-injector device can only be configured to carry the specific volume of the medicament and fail to provide provisions for varying the volume of medicament without altering the constructional or operational features of various sub-components of the auto-injector device.

Hence, this may lead to a substantial reduction in the overall reusability of the auto-injector device for administering different types and doses of medicaments. Further, the type and dose of medicaments to be administered may act as design constraints for the manufacturing of the auto-injector devices. For instance, sub-components of the auto-injector devices are required to be manufactured based on the type of medicament and a corresponding dose of such medicament to be administered through the auto-injector device. This may result in a substantial increase in the overall manufacturing cost of the auto-injector device.

### SUMMARY

This summary is provided to introduce a selection of concepts, in a simplified format, that are further described in the detailed description of the invention. This summary is neither intended to identify key or essential inventive concepts of the invention and nor is it intended for determining the scope of the invention.

In an embodiment of the present disclosure, an auto-injector device for delivering medicaments is disclosed. The auto-injector device includes a syringe assembly having a needle adapted to expel medicaments from the syringe assembly. The syringe assembly includes a barrel adapted to hold the medicaments and to be coupled to the needle. A flow of medicaments is directed out of the barrel through the needle. The syringe assembly includes a plunger positioned coaxially with respect to the barrel and adapted to reciprocate within the barrel to expel the medicaments from the barrel. Further, the syringe assembly includes a locking member adapted to be engaged with the plunger to hold the plunger in a position with respect to the barrel. The position of the plunger with respect to the barrel is adapted to be adjusted to vary a volume of medicaments to be expelled from the barrel. The position of the plunger is adjusted by engaging the locking member with the plunger at one of a plurality of positions along a length of the plunger.

To further clarify advantages and features of the present invention, a more particular description of the invention will be rendered by reference to specific embodiments thereof, which is illustrated in the appended drawings. It is appreciated that these drawings depict only typical embodiments of the invention and are therefore not to be considered limiting of its scope. The invention will be described and explained with additional specificity and detail with the accompanying drawings.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 illustrates an auto-injector device and steps involved in the implementation of the auto-injector device for delivering medicament, according to an embodiment of the present disclosure;

FIG. 2a illustrates a perspective view of the auto-injector device, according to an embodiment of the present disclosure;

FIG. 2b illustrates an exploded view of the auto-injector device, according to an embodiment of the present disclosure;

FIGS. 3a and 3b illustrate sectional views of the auto-injector device, according to an embodiment of the present disclosure;

FIG. 4 illustrates a perspective view of a plunger of the auto-injector device, according to an embodiment of the present disclosure;

FIGS. 5a-5c illustrate operation of the auto-injector device for delivering medicaments, according to an embodiment of the present disclosure; and

FIG. 6 illustrates an enlarged sectional view of a portion A of the auto-injector device as shown in FIG. 5c, according to an embodiment of the present disclosure.

Further, skilled artisans will appreciate that elements in the drawings are illustrated for simplicity and may not have been necessarily been drawn to scale. For example, the flow charts illustrate the method in terms of the most prominent steps involved to help to improve understanding of aspects of the present invention. Furthermore, in terms of the construction of the device, one or more components of the device may have been represented in the drawings by conventional symbols, and the drawings may show only those specific details that are pertinent to understanding the embodiments of the present invention so as not to obscure the drawings with details that will be readily apparent to those of ordinary skill in the art having benefit of the description herein.

### DETAILED DESCRIPTION OF FIGURES

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates. Unless otherwise defined, all technical and scientific terms used herein have the same meaning as commonly understood by one of ordinary skilled in the art to which this invention belongs. The system, methods, and examples provided herein are illustrative only and not intended to be limiting.

The term “some” as used herein is defined as “none, or one, or more than one, or all.” Accordingly, the terms “none,” “one,” “more than one,” “more than one, but not all” or “all” would all fall under the definition of “some.” The term “some embodiments” may refer to no embodiments or to one embodiment or to several embodiments or to all embodiments. Accordingly, the term “some embodiments” is defined as meaning “no embodiment, or one embodiment, or more than one embodiment, or all embodiments.”

The terminology and structure employed herein is for describing, teaching and illuminating some embodiments and their specific features and elements and does not limit, restrict or reduce the spirit and scope of the claims or their equivalents.

More specifically, any terms used herein such as but not limited to “includes,” “comprises,” “has,” “consists,” and grammatical variants thereof do NOT specify an exact limitation or restriction and certainly do NOT exclude the possible addition of one or more features or elements, unless otherwise stated, and furthermore must NOT be taken to exclude the possible removal of one or more of the listed features and elements, unless otherwise stated with the limiting language “MUST comprise” or “NEEDS TO include.”

Whether or not a certain feature or element was limited to being used only once, either way it may still be referred to as “one or more features” or “one or more elements” or “at least one feature” or “at least one element.” Furthermore, the use of the terms “one or more” or “at least one” feature or element do NOT preclude there being none of that feature or element, unless otherwise specified by limiting language such as “there NEEDS to be one or more . . . ” or “one or more element is REQUIRED.”

Unless otherwise defined, all terms, and especially any technical and/or scientific terms, used herein may be taken to have the same meaning as commonly understood by one having an ordinary skill in the art.

Reference is made herein to some “embodiments.” It should be understood that an embodiment is an example of a possible implementation of any features and/or elements presented in the attached claims. Some embodiments have been described for the purpose of illuminating one or more of the potential ways in which the specific features and/or elements of the attached claims fulfil the requirements of uniqueness, utility and non-obviousness.

Use of the phrases and/or terms such as but not limited to “a first embodiment,” “a further embodiment,” “an alternate embodiment,” “one embodiment,” “an embodiment,” “multiple embodiments,” “some embodiments,” “other embodiments,” “further embodiment”, “furthermore embodiment”, “additional embodiment” or variants thereof do NOT necessarily refer to the same embodiments. Unless otherwise specified, one or more particular features and/or elements described in connection with one or more embodiments may be found in one embodiment, or may be found in more than one embodiment, or may be found in all embodiments, or may be found in no embodiments. Although one or more features and/or elements may be described herein in the context of only a single embodiment, or alternatively in the context of more than one embodiment, or further alternatively in the context of all embodiments, the features and/or elements may instead be provided separately or in any appropriate combination or not at all. Conversely, any features and/or elements described in the context of separate embodiments may alternatively be realized as existing together in the context of a single embodiment.

Any particular and all details set forth herein are used in the context of some embodiments and therefore should NOT be necessarily taken as limiting factors to the attached claims. The attached claims and their legal equivalents can be realized in the context of embodiments other than the ones used as illustrative examples in the description below.

Embodiments of the present invention will be described below in detail with reference to the accompanying drawings.

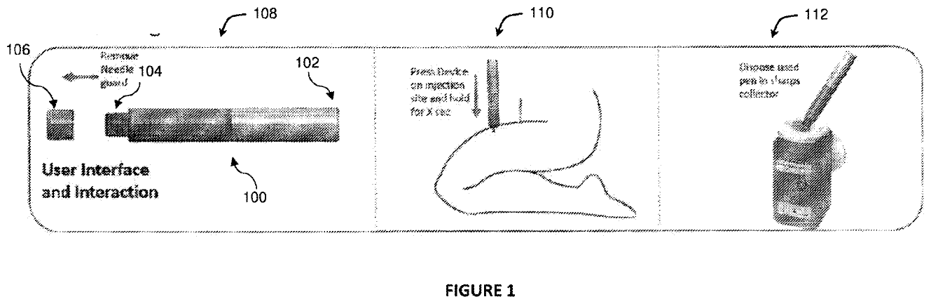

FIG. 1 illustrates an auto-injector device 100 and steps involved in the implementation of the auto-injector device 100 for delivering medicament, according to an embodiment of the present disclosure. In an embodiment, the auto-injector device 100 may be employed for delivering medicaments, interchangeably be referred to as drugs, to a patient. The auto-injector device 100 may include a proximal end 102 and a distal end 104. Further, the auto-injector device 100 may include a cap 106 removably attached to the distal end 104 of the auto-injector device 100. The cap 106 may be provided to protect a needle of the auto-injector device 100 and to restrict the ingress of any contaminants within the auto-injector device 100.

In order to implement the auto-injector device 100 for medication, referring to FIG. 1, at step 108, the cap 106 of the auto-injector device 100 may be removed for delivering medicaments to the patient. Subsequently, at step 110, the distal end 104 of the auto-injector device 100 may be placed on a site of injection corresponding to a subcutaneous (sub-Q) region, on the patient for delivering the medicaments. Upon placing the auto-injector device 100 on the site, the auto-injector device 100 may be pressed in a direction towards the site of injection and held for a predefined period of time for delivering the medicaments to the patient through the site. Subsequently, at step 112, the auto-injector device 100 may be removed from the site of injection and disposed in sharp collectors.

Constructional and operational details of the auto-injector device 100 are explained in detail in the subsequent sections of the present disclosure.

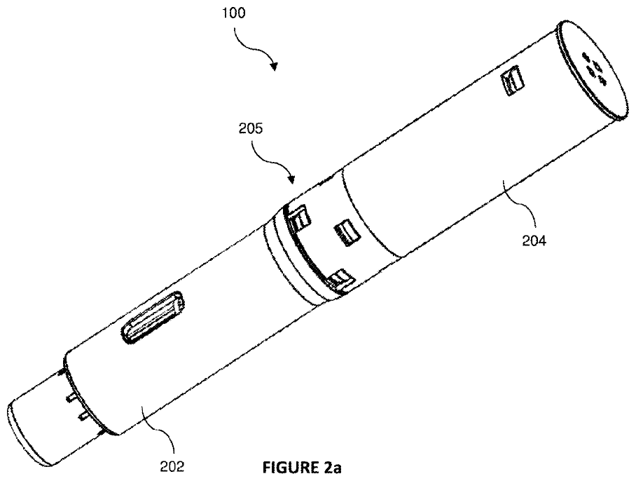

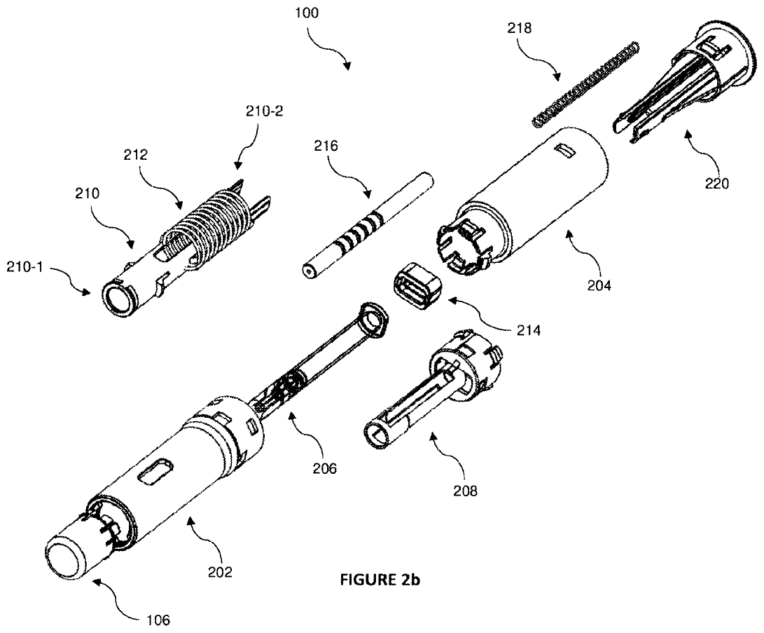

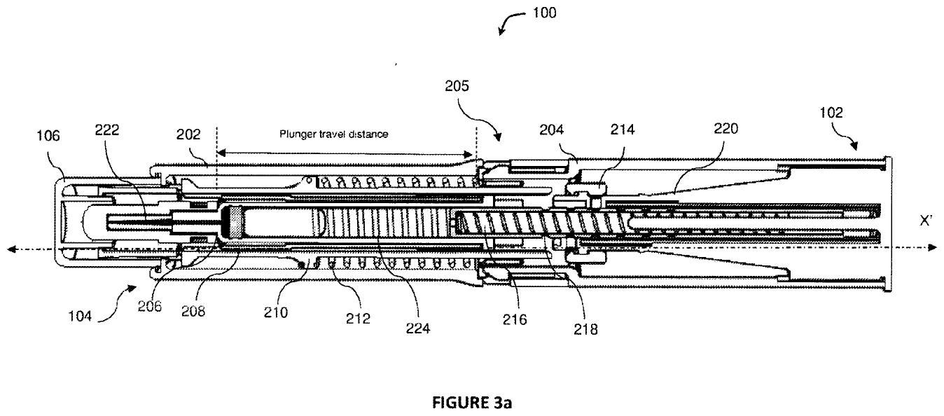

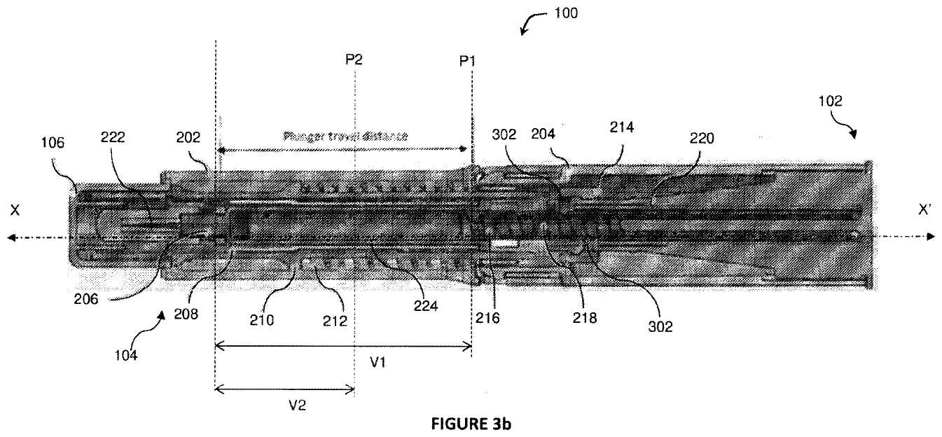

FIG. 2a illustrates a perspective view of the auto-injector device, according to an embodiment of the present disclosure. FIG. 2b illustrates an exploded view of the auto-injector device 100, according to an embodiment of the present disclosure. FIGS. 3a and 3b illustrate sectional views of the auto-injector device 100, according to an embodiment of the present disclosure. Referring to FIG. 2a, FIG. 2b, and FIG. 3a, in an embodiment, the auto-injector device 100 may include a first body member 202 and a second body member 204. The first body member 202 and the second body member 204 may be adapted to be coupled to each other in order to define a housing 205 for accommodating various sub-components of the auto-injector device 100. In an embodiment, the first body member 202 and the second body member 204 may be coupled with each other through snap locks, without departing from the scope of the present disclosure.

Further, the auto-injector device 100 may include a syringe assembly 206, a Pre-filled Syringe (PFS) holder 208, a needle guard 210, a needle guard spring 212, a locking member 214, a plunger 216, a plunger spring 218, and spring release arms 220. In an embodiment, the syringe assembly 206 may include, but is not limited to, a needle 222 and a barrel 224 adapted to be coupled to the needle 222. The barrel 224 may be adapted to hold the medicaments. The barrel 224 may include a front portion and a rear portion distal to the front portion. The front portion may be adapted to be coupled to the needle 222 and the rear portion may be adapted to movably receive the plunger 216.

Further, the needle 222 may be adapted to expel medicaments from the syringe assembly 206. The needle 222 may be adapted to be coupled to the front portion of the barrel 224 such that a flow of the medicaments may be directed out of the barrel 224 through the needle 222 during an operation of the auto-injector device 100. Further, the PFS holder 208 may be adapted to hold the syringe assembly 206 within the housing 205 of the auto-injector device 100.

In an embodiment, the needle guard 210 may be adapted to accommodate the PFS holder 208 along with the syringe assembly 206 in the auto-injector device 100. The needle guard 210 may include a proximal end 210-1 and a distal end 210-2. Further, the needle guard spring 212 may be adapted to be coupled to the needle guard 210. The needle guard spring 212 may be provided to allow a resilient movement of the needle guard 210 in a direction along an axis X-X′ of the auto-injector device 100 during the operation of the auto-injector device 100.

Further, the spring release arms 220 may be positioned at the proximal end 102 of the auto-injector device 100. In an embodiment, the spring release arm 220 may be adapted to be coupled to the second body member 204. The spring release arms 220 may be adapted to support the plunger 216 and the plunger spring 218 within the auto-injector device 100. The plunger 216 may be positioned coaxially with respect to the barrel 224. The plunger 216 may be adapted to reciprocate within the barrel 224 to expel the medicaments and to draw the medicaments in the barrel 224. The plunger 216 may be adapted to be moved along the axis X-X′ of the auto-injector device 100. The plunger 216 may be adapted to reciprocate within the barrel 224 of the syringe assembly 206 for expelling the medicaments from the syringe assembly 206 and for drawing the medicaments within the barrel 224.

Further, the plunger spring 218 may be provided to allow resilient movement of the plunger 216 within the barrel 224 in the direction along the axis X-X′. In particular, during the operation of the auto-injector device 100, the plunger spring 218 may be adapted to resiliently push the plunger 216 within the barrel 224 in order to expel the medicaments from the barrel 224 of the syringe assembly 206. The plunger spring 218 may be adapted to be compressed between a head portion of the plunger 216 and the locking member 214.

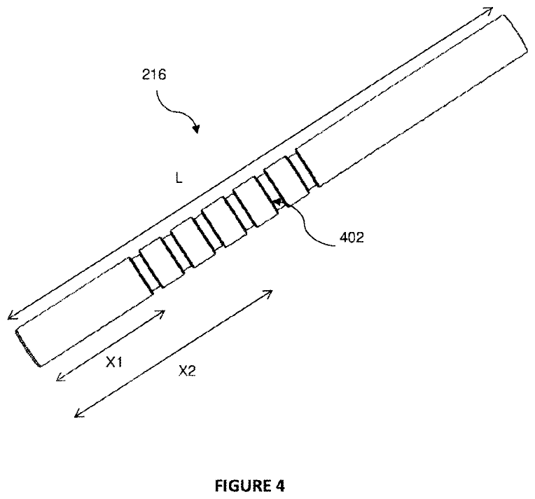

FIG. 4 illustrates a perspective view of the plunger 216 of the auto-injector device 100, according to an embodiment of the present disclosure. In an embodiment, the locking member 214 may be adapted to be engaged with the plunger 216 to hold the plunger in a position with respect to the barrel 224. Further, the locking member 214 may be adapted to hold the spring release arm 220 in a locking position to ensure engagement of the spring release arm 220 with the plunger 216. In an embodiment, the locking member 214 may be adapted to circumscribe at least a portion of the spring release arm 220 to eliminate flexing of the spring release arm 220 in a radially outward direction with respect to the plunger 216 and thereby, ensuring engagement of the spring release arm 220 with the plunger 216.

Referring to FIG. 3a, FIG. 3b, and FIG. 4, the plunger 216 may include an engaging portion 402 extending along a length L of the plunger 216. In an embodiment, the engaging portion 402 may be embodied as one of spiral grooves and circular grooves, without departing from the scope of the present disclosure. The locking member 214 may be adapted to be engaged with the engaging portion 402 of the plunger 216 to hold the plunger 216 in the position within the auto-injector device 100. In particular, the locking member 214 may engage at one of the plurality of positions in the engaging portion 402 along the length L of the plunger 216.

The position of the plunger 216 with respect to the barrel 224 may be adapted to be adjusted to vary a volume of medicaments to be expelled from the barrel 224. The position of the plunger 216 may be adjusted by engaging the locking member 214 with the plunger 216 at one of a plurality of positions along the length L of the plunger 216. In an embodiment, the locking member 214 may include locking teeth 302 adapted to be engaged with the engaging portion 402 of the plunger 216. The locking member 214 may be operable between an engaged position and a disengaged position.

In an embodiment, the needle guard 210 may be adapted to move the locking member 214 from the engaged position to the disengaged position. In the engaged position, the locking teeth 302 may be engaged with the engaging portion 402 to hold the plunger 216 in a position away from the distal end 104 of the auto-injector device 100. In such a position, the plunger spring 218 may be compressed between the head portion of the plunger 216 and the locking member 214. Further, in the disengaged position, the needle guard 210 may move the locking member 214 towards the proximal end 102 of the auto-injector device 100. Owing to such movement of the locking member 214, the locking teeth 302 may be disengaged from the engaging portion 402 of the plunger 216. Subsequently, the plunger spring 218 may push the plunger 216 towards the distal end 104 of the auto-injector device 100 within the barrel 224 to expel the medicaments from the syringe assembly 206.

As mentioned earlier, the position of the plunger 216 with respect to the barrel 224 within the auto-injector device 100 may be adjustable for delivering different volumes of medicaments from the auto-injector device 100. Referring to FIG. 3b and FIG. 4, in one instance, the locking member 214 may be engaged with the engaging portion 402 at a distance X1 with respect to the length L of the plunger 216. In such an instance, the plunger 216 may be held at a position P1 with respect to the front portion 210-1 of the barrel 224 within the auto-injector device 100.

Owing to such a position of the plunger 216, the barrel 224 may hold a volume V1, i.e., a maximum volume, of medicaments for dispensing through the needle 222 during the operation of the auto-injector device 100. In another instance, the locking member 214 may be engaged with the engaging portion 402 at a distance X2 with respect to the length L of the plunger 216. In such an instance, the plunger 216 may be held at a position P2 with respect to the front portion 210-1 of the barrel 224 within the auto-injector device 100. Owing to such a position of the plunger 216, the barrel 224 may hold a volume V2 of medicaments for dispensing through the needle 222.

Although, in the illustrated embodiment, operation of the plunger 216 and the locking member 214 is explained with respect to the positions P1 and P2 of the plunger 216 within the auto-injector device 100. As would be appreciated by the person skilled in the art, the plunger 216 can be locked at a plurality of positions by the locking member 214 for varying the volume of the medicaments to be delivered from the auto-injector device 100 as per the requirement, without departing from the scope of the present disclosure.

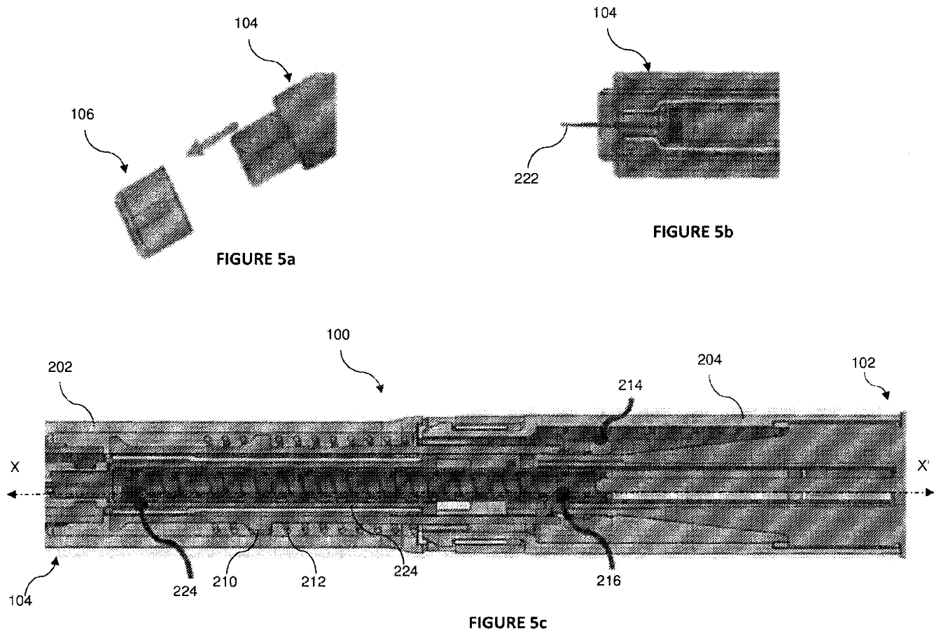

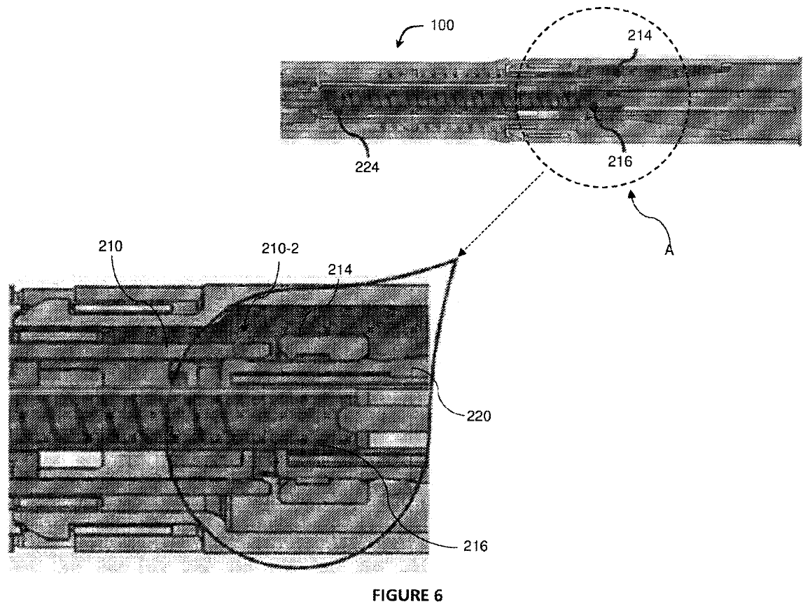

FIGS. 5a-5c illustrate operation of the auto-injector device 100 for delivering medicaments, according to an embodiment of the present disclosure. FIG. 6 illustrates an enlarged sectional view of a portion A of the auto-injector device 100 as shown in FIG. 5c, according to an embodiment of the present disclosure. Firstly, in order to deliver the medicaments to the sub-Q region of the patient through the auto-injector device 100, the cap 106 may be removed from the distal end 104 of the auto-injector device 100 to expose the needle 222 of the syringe assembly 206.

Further, referring to FIG. 5b, the distal end 104 of auto-injector device 100 may be placed on the site of injection i.e., like skin, corresponding to the sub-Q region of the patient. In an embodiment, the site of injection may be selected based on various parameters, such as a medical prescription, type of medication, and volume of medicament to be injected, without departing from the scope of the present disclosure. Subsequently, the auto-injector device 100 may be pressed against the skin such that the needle guard 210 is pressed against the skin which results in movement of the needle guard 210 in a direction towards the proximal end 102 of the auto-injector device 100. Owing to such movement of the needle guard 210, the needle 222 may be inserted in the sub-Q region of the patient for delivering the medicaments. Further, upon injecting the medicaments, the auto-injector device 100 may be moved away from the skin which results in the movement of the needle guard 210 to cover the needle 222 of the auto-injector device 100. This substantially eliminates probable injuries which might cause due to exposed needle while withdrawing the auto-injector device 100 from the injection site.

Referring to FIG. 5c and FIG. 6, the movement of the needle guard 210 may push the locking member 214 towards the proximal end 102 of the auto-injector device 100. Such movement of the locking member 214 may result in disengagement of the locking teeth 302 with the engaging portion 402 of the plunger 216. In particular, the locking teeth 302 may move in a lateral direction away from the engaging portion 402 and thereby, unlocking a movement of the plunger 216 under a resilient force of the plunger spring 218. Subsequently, the plunger 216 may move towards the distal end 104 of the auto-injector device 100 within the barrel 224 under the resilient force of the plunger spring 218. The plunger 216 may push the medicament held within the barrel 224 for delivering the medicament to the sub-Q region of the patient through the needle 222 of the syringe assembly 206. In an embodiment, a rubber stopper (not shown) may be attached to the plunger 216 and adapted to be moved along with the plunger 216 within the barrel 224 to expel the drug held within the barrel 224 of the syringe assembly 206. The rubber stopper may be adapted to be inserted in the barrel 224 in a manner that the rubber stopper may act as a sealing member to prevent egress of the drug from one end of the barrel 224. The rubber stopper may be moved along with the plunger 216 to expel the drug from the barrel 224 through the needle 222 positioned at another end of the barrel 224.

As would be gathered, the auto-injector device 100 of the present disclosure includes the locking member 214 adapted to lock the plunger 216 at different positions in order to administer different volumes of medicaments through the auto-injector device 100. The plunger 216 may include the engaging portion 402 extending along the length L of the plunger 216. The locking member 214 may be engaged with the engaging portion 402 to hold the plunger 216 in a pre-loaded position for expelling the medicaments from the syringe assembly 206. The locking member 214 can be engaged with the engaging portion 402 at different positions along the length L of the plunger 216 in order to vary the volume of medicament to be administered through the auto-injector device 100.

Therefore, the auto-injector device 100 can be re-used for different types and doses of medicaments without incorporating any alteration to the sub-components of the auto-injector device 100. In particular, this may eliminate the requirement for manufacturing different sub-components of the auto-injector device 100 for administering different types and doses of medicaments. Further, this substantially eliminates the overall manufacturing cost of the auto-injector device 100. Therefore, the auto-injector device 100 of the present disclosure is flexible in implementation, compact, cost-effective, convenient, and has a wide range of applications.

The auto-injector device 100 described herein may be used for delivering different therapeutic compounds such as drugs and biologics, including but not limited to, antibodies, antisense, RNA interference, gene therapy, primary and embryonic stem cells, vaccines, and combinations thereof. For instance, the embodiments described herein may be utilized in combination with known monoclonal antibodies including but not limited to Abciximab, Abituzumab, Abrilumab, Actoxumab, Adalimumab, Adecatumumab, Aducanumab, Afasevikumab, Afelimomab, Afutuzumab, Alacizumab pegol, ALD518, ALD403, Alemtuzumab, Alirocumab, Altumomab pentetate, Amatuximab, AMG 334, Anatumomab mafenatox, Anetumab ravtansine, Anifrolumab, Anrukinzumab, Apolizumab, Arcitumomab, Ascrinvacumab, Aselizumab, Atezolizumab, Atinumab, Atlizumab, Atorolimumab, Avelumab, Bapineuzumab, Basiliximab, Bavituximab, Bectumomab, Begelomab, Belimumab, Benralizumab, Bertilimumab, Besilesomab, Bevacizumab, Bezlotoxumab, Biciromab, Bimagrumab, Bimekizumab, Bivatuzumab mertansine, Bleselumab, Blinatumomab, Blontuvetmab, Blosozumab, Bococizumab, Brazikumab, Brentuximab vedotin, Briakinumab, Brodalumab, Brolucizumab, Brontictuzumab, Burosumab, Cabiralizumab, Canakinumab, Cantuzumab mertansine, Cantuzumab ravtansine, Caplacizumab, Capromab pendetide, Casirivimab, Carlumab, Carotuximab, Catumaxomab, cBR96-doxorubicin immunoconjugate, Cedelizumab, Cergutuzumab amunaleukin, Certolizumab pegol, Cetuximab, Citatuzumab bogatox, Cixutumumab, Clazakizumab, Clenoliximab, Clivatuzumab tetraxetan, Codrituzumab, Coltuximab ravtansine, Conatumumab, Concizumab, CR6261, Crenezumab, Crotedumab, Dacetuzumab, Daclizumab, Dalotuzumab, Dapirolizumab pegol, Daratumumab, Dectrekumab, Demcizumab, Denintuzumab mafodotin, Denosumab, Depatuxizumab mafodotin, Derlotuximab biotin, Detumomab, Dinutuximab, Diridavumab, Domagrozumab, Dorlimomab aritox, Drozitumab, Duligotumab, Dupilumab, Durvalumab, Dusigitumab, Ecromeximab, Eculizumab, Edobacomab, Edrecolomab, Efalizumab, Efungumab, Eldelumab, Elgemtumab, Elotuzumab, Elsilimomab, Emactuzumab, Emibetuzumab, Emicizumab, Enavatuzumab, Enfortumab vedotin, Enlimomab pegol, Enoblituzumab, Enokizumab, Enoticumab, Ensituximab, Epitumomab cituxetan, Epratuzumab, Erenumab, Erlizumab, Ertumaxomab, Etaracizumab, Etrolizumab, Evinacumab, Evolocumab, Exbivirumab, Fanolesomab, Faralimomab, Farletuzumab, Fasinumab, FBTA05, Felvizumab, Fezakinumab, Fibatuzumab, Ficlatuzumab, Figitumumab, Firivumab, Flanvotumab, Fletikumab, Fontolizumab, Foralumab, Foravirumab, Fresolimumab, Fulranumab, Futuximab, Galcanezumab, Galiximab, Ganitumab, Gantenerumab, Gavilimomab, Gemtuzumab ozogamicin, Gevokizumab, Girentuximab, Glembatumumab vedotin, Golimumab, Gomiliximab, Guselkumab, Ibalizumab, Ibritumomab tiuxetan, Icrucumab, Idarucizumab, Igovomab, IMA-638, IMAB362, Imalumab, Imciromab, Imdevimab, Imgatuzumab, Inclacumab, Indatuximab ravtansine, Indusatumab vedotin, Inebilizumab, Infliximab, Inolimomab, Inotuzumab ozogamicin, Intetumumab, Ipilimumab, Iratumumab, Isatuximab, Itolizumab, Ixekizumab, Keliximab, Labetuzumab, Lambrolizumab, Lampalizumab, Lanadelumab, Landogrozumab, Laprituximab emtansine, LBR-101/PF0442g7429, Lebrikizumab, Lemalesomab, Lendalizumab, Lenzilumab, Lerdelimumab, Lexatumumab, Libivirumab, Lifastuzumab vedotin, Ligelizumab, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, LY2951742, Mapatumumab, Margetuximab, Maslimomab, Matuzumab, Mavrilimumab, Mepolizumab, Metelimumab, Milatuzumab, Minretumomab, Mirvetuximab soravtansine, Mitumomab, Mogamulizumab, Monalizumab, Morolimumab, Motavizumab, Moxetumomab pasudotox, Muromonab-CD3, Nacolomab tafenatox, Nam ilumab, Naptumomab estafenatox, Naratuximab emtansine, Narnatumab, Natalizumab, Navicixizumab, Navivumab, Nebacumab, Necitumumab, Nemolizumab, Nerelimomab, Nesvacumab, Nimotuzumab, Nivolumab, Nofetumomab merpentan, Obiltoxaximab, Obinutuzumab, Ocaratuzumab, Ocrelizumab, Odulimomab, Ofatumumab, Olaratumab, Olokizumab, Omalizumab, Onartuzumab, Ontuxizumab, Opicinumab, Oportuzumab monatox, Oregovomab, Orticumab, Otelixizumab, Otlertuzumab, Oxelumab, Ozanezumab, Ozoralizumab, Pagibaximab, Palivizumab, Pamrevlumab, Panitumumab, Pankomab, Panobacumab, Parsatuzumab, Pascolizumab, Pasotuxizumab, Pateclizumab, Patritumab, Pembrolizumab, Pemtumomab, Perakizumab, Pertuzumab, Pexelizumab, Pidilizumab, Pinatuzumab vedotin, Pintumomab, Placulumab, Plozalizumab, Pogalizumab, Polatuzumab vedotin, Ponezumab, Prezalizumab, Priliximab, Pritoxaximab, Pritumumab, PRO 140, Quilizumab, Racotumomab, Radretumab, Rafivirumab, Ralpancizumab, Ramucirumab, Ranibizumab, Raxibacumab, Refanezumab, Regavirumab, Reslizumab, Rilotumumab, Rinucumab, Risankizumab, Rituximab, Rivabazumab pegol, Robatumumab, Roledumab, Romosozumab, Rontalizumab, Rovalpituzumab tesirine, Rovelizumab, Ruplizumab, Sacituzumab govitecan, Samalizumab, Sapelizumab, Sarilumab, Satumomab pendetide, Secukinumab, Seribantumab, Setoxaximab, Sevirumab, SGN-CD19A, SGN-CD33A, Sibrotuzumab, Sifalimumab, Siltuximab, Simtuzumab, Siplizumab, Sirukumab, Sofituzumab vedotin, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tamtuvetmab, Tanezumab, Taplitumomab paptox, Tarextumab, Tefibazumab, Telimomab aritox, Tenatumomab, Teneliximab, Teplizumab, Teprotumumab, Tesidolumab, Tetulomab, Tezepelumab, TGN1412, Ticilimumab, Tigatuzumab, Tildrakizumab, Timolumab, Tisotumab vedotin, TNX-650, Tocilizumab, Toralizumab, Tosatoxumab, Tositumomab, Tovetumab, Tralokinumab, Trastuzumab, Trastuzumab emtansine, TRB S07, Tregalizumab, Tremelimumab, Trevogrumab, Tucotuzumab celmoleukin, Tuvirumab, Ublituximab, Ulocuplumab, Urelumab, Urtoxazumab, Ustekinumab, Utomilumab, Vadastuximab talirine, Vandortuzumab vedotin, Vantictumab, Vanucizumab, Vapaliximab, Varlilumab, Vatelizumab, Vedolizumab, Veltuzumab, Vepalimomab, Vesencumab, Visilizumab, Vobarilizumab, Volociximab, Vorsetuzumab mafodotin, Votumumab, Xentuzumab, Zalutumumab, Zanolimumab, Zatuximab, Ziralimumab, and Zolimomab aritox or combinations thereof.

While specific language has been used to describe the present subject matter, any limitations arising on account thereto, are not intended. As would be apparent to a person in the art, various working modifications may be made to the method in order to implement the inventive concept as taught herein. The drawings and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment.

1-10. (canceled)

11. An auto-injector device comprising:
a syringe assembly comprising:
a needle adapted to expel medicaments from the syringe assembly; and
a barrel adapted to hold the medicaments and to be coupled to the needle, wherein a flow of medicaments is directed out of the barrel through the needle;
a plunger positioned coaxially with respect to the barrel and adapted to reciprocate within the barrel to expel the medicaments; and
a locking member adapted to be engaged with the plunger to hold the plunger in a position with respect to the barrel,
wherein the position of the plunger with respect to the barrel is adapted to be adjusted to vary a volume of medicaments to be expelled from the barrel, the position of the plunger is adjusted by engaging the locking member with the plunger at one of a plurality of positions along a length (L) of the plunger.

12. The auto-injector device as claimed in claim 11, further comprising a first body member and a second body member, wherein the first body member and the second body member are adapted to be coupled to each other to define a housing therein.

13. The auto-injector device as claimed in claim 12, further comprising a Pre-filled Syringe (PFS) holder adapted to hold the syringe assembly within the housing.

14. The auto-injector device as claimed in claim 11, wherein the plunger includes an engaging portion, extending along the length (L) of the plunger, wherein the locking member engages at one of the plurality of positions in the engaging portion along the length (L).

15. The auto-injector device as claimed in claim 11, further comprising a spring release arm positioned at a proximal end of the auto-injector device and adapted to support the plunger and a plunger spring.

16. The auto-injector device as claimed in claim 15, wherein the locking member includes locking teeth adapted to be engaged with the engaging portion of the plunger, wherein the locking member is adapted to hold the spring release arm in a locking position to ensure engagement of the spring release arm with the plunger.

17. The auto-injector device as claimed in claim 11, further comprising a needle guard adapted to move the locking member from an engaged position to a disengaged position.

18. The auto-injector device as claimed in claim 17, further comprising a needle guard spring adapted to be coupled to the needle guard, wherein the needle guard spring is adapted to allow a resilient movement of the needle guard.

19. The auto-injector device as claimed in claim 17, wherein:
in the engaged position, the locking teeth engages with the engaging portion to hold the plunger in the position away from a distal end of the auto injector device; and
in the disengaged position, the needle guard moves the locking member towards the proximal end of the auto-injector device to disengage the locking teeth from the engaging portion.

20. The auto-injector device as claimed in claim 11, further comprising:
a plunger spring adapted to be compressed between a head portion of the plunger and the locking member, wherein the plunger spring is adapted to resiliently push the plunger within the barrel to expel the medicaments from the barrel.
